(19) United States
(12) Patent Application Publication
(10) Pub. No.: US 2024/0191182 A1
(43) Pub. Date: Jun. 13, 2024

(54) ADHESION IMPROVER CONTAINING EDIBLE AND NON-LETHAL ANIMAL-DERIVED COMPONENT

(71) Applicant: NITTO DENKO CORPORATION, Ibaraki-shi, Osaka (JP)

(72) Inventors: Yuka SEKIGUCHI, Ibaraki-shi, Osaka (JP); Tomoko SUDO, Ibaraki-shi, Osaka (JP); Tomoaki HISHIKI, Ibaraki-shi, Osaka (JP); Takahisa KONISHI, Ibaraki-shi, Osaka (JP); Yasutaka KAWASAKI, Ibaraki-shi, Osaka (JP)

(21) Appl. No.: 18/285,061

(22) PCT Filed: Mar. 31, 2022
(86) PCT No.: PCT/JP2022/016597

§ 371 Date: Sep. 29, 2023

(30) Foreign Application Priority Data
Mar. 31, 2021 (JP) 2021-061251

(51) Int. Cl. C12N 5/00

(52) U.S. Cl. CPC C12N 5/0068; C12N 2533/50; C12N 2533/74; C12N 2533/78; C12N 2539/00

(57) ABSTRACT

The purpose of the present invention is to provide an adhesion improver that is to be used for an edible base material for cell culturing and contains an edible and non-lethal animal-derived component, a cell culturing scaffold material containing the adhesion improver, a tissue body containing the cell culturing scaffold material, etc.

A cell culturing scaffold material suitable for the production of a cultured meat can be produced by applying an adhesion improver containing an edible and non-lethal animal-derived component to an edible base material for cell culturing.

### TECHNICAL FIELD

The present invention relates to an adhesion improver that is to be used for an edible base material for cell culturing and contains an edible and non-lethal animal-derived component, a cell culturing scaffold material containing the adhesion improver, and a tissue body containing the cell culturing scaffold material.

### BACKGROUND ART

In recent years, the demand for meat is expected to increase as the world population grows. In order to meet future demand for meat, it is not enough to simply increase manufacturing efficiency to increase manufacturing volume of conventional protein sources and it is indispensable to develop new protein sources. The new protein sources include plant-based meat manufactured from plants, meat manufactured from insects, and cultured meat manufactured by culturing microorganisms or cells themselves.

“Cultured meat” refers to a meat produced by culturing muscle cells using regenerative medicine technology, which is also called as “cultured meat” or “clean meat. One of the advantages of the cultured meat is safety. For example, in a process of manufacturing and processing meat, there is always some risks of contamination of pathogenic bacteria that may cause food poisoning. However, the cultured meat is cultured under nearly sterile condition, so the risk of contamination of pathogenic bacteria is lower. Using the cultured meat can reduce greenhouse gas emissions by 96% compared to conventional producing methods, in addition to reducing the cost required for processing process according to a result of study, which attracts attention from the environmental perspective.

During producing of cultured meat, it is desirable not to use a material obtained by killing animal such as gelatin and collagen, for its purpose. In addition, it is required to culture muscle cells three-dimensionally using a scaffold material in order to produce cultured meat controlled to have a desired shape and texture, such as steak meat, sashimi, or fillet. However, such scaffold material often has poor cell adhesion and cells may not grow successfully. In wound dressing and graft, those in which peptide is used (Patent Document 1) and silk fiber is used (Patent Document 2) are known to improve adhesion of cells and them.

In Patent Document 1, a wound dressing in which a peptide containing the smallest amino acid sequence that exhibits cell adhesion signal to improve cell adhesion is included, but this peptide is cultured by E. coli and is not edible, although it is a non-lethal animal-derived component. Further, in Patent Document 1, it is described that serum-derived proteins such as albumin and milk-derived proteins such as casein may be used in production of the wound dressing, but these are used as a blocking agent and not for improving cell adhesion.

In addition, in Patent Document 2, a fiber inducer to enhance adhesiveness of medical graft to tissue is described. This fiber inducer is composed of silk fiber, etc., and the silk fiber is a lethal animal-derived component. Further, casein which is milk-derived protein is used for production of the medical graft described in the Patent Document 2, but it is used as a biodegradable polymer suitable for delivery of fiber-inducing agent, not as a fiber-inducing agent.

### PRIOR ART REFERENCES

### Patent References

### Patent Document 1:

Japanese Unexamined Patent Application Publication No. 2003-89648

### Patent Document 2:

Japanese Unexamined Patent Application Publication (Translation of PCT Application) No. 2007-513083

### SUMMARY OF INVENTION

### Problems to be Solved by Invention

The purpose of the present invention is to provide an adhesion improver that is to be used for an edible base material for cell culturing and contains an edible and non-lethal animal-derived component, a cell culturing scaffold material containing the adhesion improver, a tissue body containing the cell culturing scaffold material, etc.

### Means of Solving Problems

During producing of cultured meat, it is desirable not to use a material obtained by killing animal such as gelatin and collagen, for its purpose. In addition, it is required to culture muscle cells three-dimensionally using a scaffold material in order to produce cultured meat controlled to have a desired shape and texture, such as steak meat, sashimi, or fillet, but such scaffold material often has poor cell adhesion and cells may not grow successfully, and enhancing cell adhesion of the scaffold material has been a problem.

The inventors have found, as a result of diligent researches to solve the aforementioned problems, that a cell culturing scaffold material suitable for producing cultured meat can be produced by applying an adhesion improver containing an edible and non-lethal animal to an edible base material for cell culturing, and further studies have been conducted to complete the present invention.

In other words, the present invention relates to:

(1) An adhesion improver containing edible and non-lethal animal-derived component that is used in an edible base material for cell culturing.

(2) The adhesion improver according to (1), wherein the edible and non-lethal animal-derived component is derived from milk or egg.

(3) The adhesion improver according to (2), wherein the milk is cow's milk.

(4) The adhesion improver according to any one of (1) to (3), wherein the edible and non-lethal animal-derived component is casein or whey.

(5) The adhesion improver according to (2), wherein the egg is a hen's egg.

(6) The adhesion improver according to any one of (1) to (2) and (5), wherein the edible and non-lethal animal-derived component is egg yolk or egg white.

(7) The adhesion improver according to any one of (1) to (6), wherein the edible base material contains alginic acid or alginate, glucomannan, or cellulose derivative.

(8) The adhesion improver according to any one of (1) to (7), wherein the edible base material is a porous material.

(9) A cell culturing scaffold material containing the edible base material for cell culturing and the adhesion improver according to any one of (1) to (8).

(10) The cell culturing scaffold material according to (9), wherein a layer containing said adhesion improver is laminated on a surface of the edible base material.

(11) The cell culturing scaffold material according to (10), wherein thickness of the layer containing said adhesion improver is 1 nm or more.

(12) A tissue body in which cells are cultured on the cell culturing scaffold material according to any one of (9) to (11).

(13) The tissue body according to (12), wherein the cell is derived from mammal, fish, or crustacean.

(14) The tissue body according to (12) or (13), wherein the tissue body is for food.

### Advantageous Effects of Invention

The adhesion improver of the present invention can improve adhesion between cell culturing base material and cells. Applying the adhesion improver of the present invention onto an edible base material in which a desired shape and texture are achieved makes it possible to produce a cell culturing scaffold material in which the desired shape and texture are achieved, and further, which makes it possible to produce a cultured meat in which a desired shape and texture are achieved.

Furthermore, the adhesion improver of the present invention can be used in a scene where cell culturing is performed, and can be used not only in the field of cultured meat, but also in the field of pharmaceutical raw material manufacture and/or chemical raw material manufacture.

### BRIEF DESCRIPTION OF DRAWINGS

FIG. 1(A) is a two-dimensional image of a sample in example 1. FIG. 1(B) is a three-dimensional image of a sample in example 1.

FIG. 2(A) is a two-dimensional image of a sample in example 2. FIG. 2(B) is a three-dimensional image of a sample in example 2.

FIG. 3(A) is a two-dimensional image of a sample in example 3. FIG. 3(B) is a three-dimensional image of a sample in example 3.

FIG. 4(A) is a two-dimensional image of a sample in example 4. FIG. 4(B) is a three-dimensional image of a sample in example 4.

FIG. 5 is a three-dimensional image of a sample in Reference Example.

FIG. 6 is a three-dimensional image of a sample in Comparative Example 1.

FIG. 7 are SEM images of specimens in Comparative Example 1, Example 7, and Examples 13.

FIG. 8 are TOF-SIMS images of specimens in Comparative Example 1 and Example 7.

FIG. 9 are SEM images of specimens in Comparative Example 1, Example 8 and Example 14.

FIG. 10 are TOF-SIMS images of specimens in Comparative Example 1, and Examples 8.

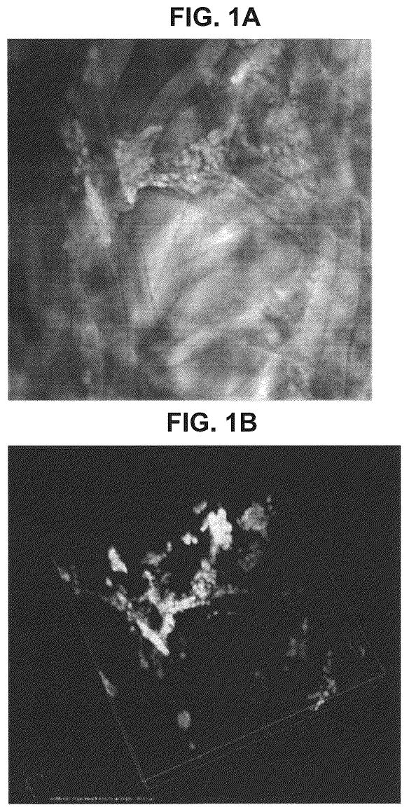

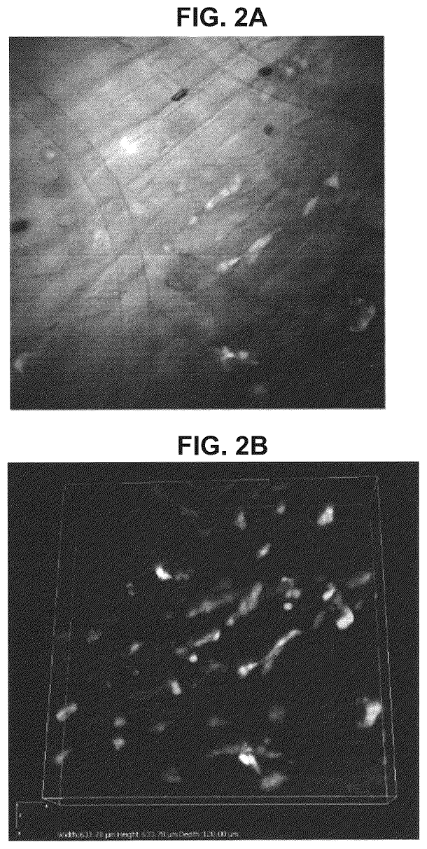

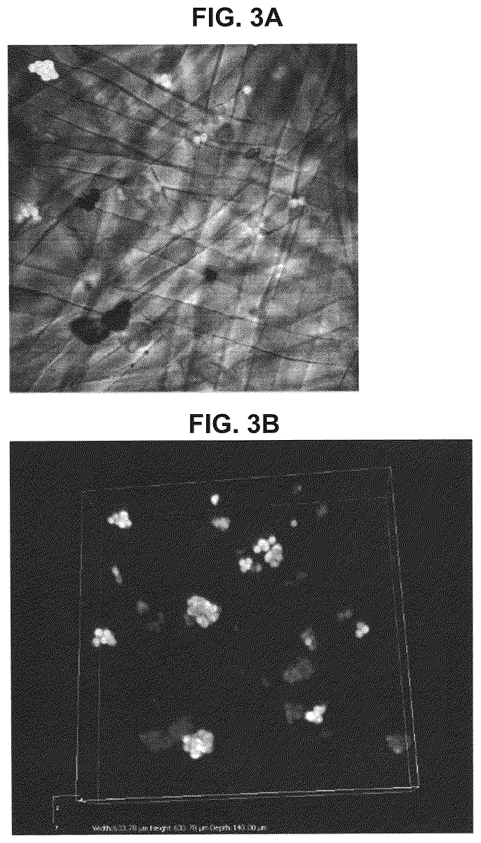

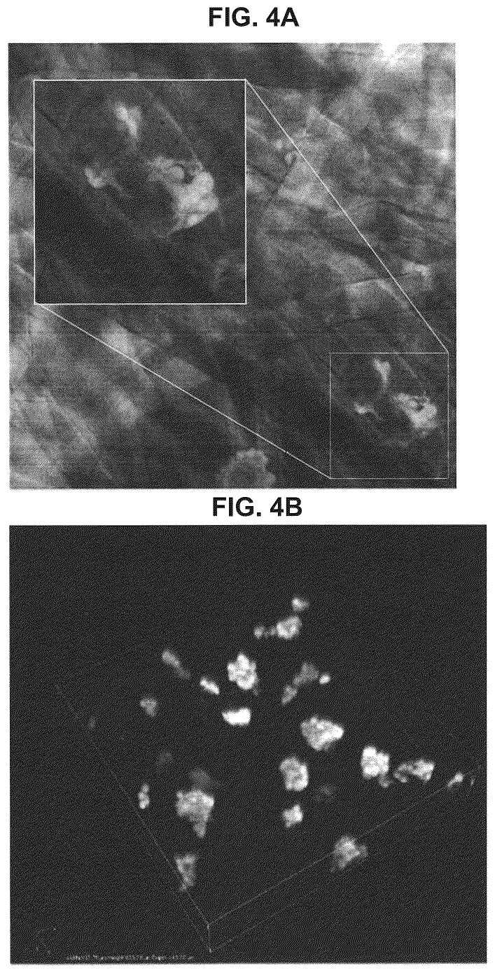

### EMBODIMENTS FOR CARRYING OUT INVENTION

The present invention is described in detail below.

Unless otherwise defined herein, all technical and scientific terms used herein have the same meanings as those ordinarily understood by those skilled in the art. All patents, applications and other publications and information referenced herein are incorporated herein by reference in their entirety. Moreover, in the event of any inconsistency between the publication referenced herein and the description herein, the description herein shall prevail.

The present invention relates to an adhesion improver containing edible and non-lethal animal-derived component used in an edible base material for cell culturing.

In the present invention, “edible” means that it can be safely ingested into a body, and that it consists only of substances, for example, that are identified as food or food additives by law, etc. of each country. So far as it is edible, it may or may not be absorbed into the body, and may or may not be digestible.

In the present invention, “edible base material for cell culturing” means a base material used for cell culturing, and that can be safely ingested into a body, and that the base material consists only of substances, for example, that are identified as food or food additives by law, etc. of each country.

In one embodiment of the present invention, the edible base material of the present invention is chewable and can be broken/shredded into smaller pieces by chewing before swallowing.

In the present invention, the edible base material with physical properties (e.g., Young's modulus, viscosity, robustness, etc.) corresponding to desired use (e.g., consumed by human adults) can be selected.

In the present invention, “non-lethal animal-derived component” refers to a component whose raw material is an animal that can be obtained without slaughtering the animal. In the present invention, the edible and non-lethal animal-derived component is not particularly limited, but is, for example, milk, egg, blood, and crop milk of the animal, and is preferably milk or egg of the animal.

In the present invention, the milk is not particularly limited, but is, for example, milk of cow, goat, sheep, water buffalo, camel, donkey, horse, reindeer, and yak, and preferably milk of cow (cow's milk).

In the present invention, non-lethal animal-derived component derived from milk is not particularly limited, but is, for example, casein, whey, milk fat, milk sugar, vitamin, and mineral, preferably casein or whey. Casein is, for example, sodium caseinate.

In the present invention, egg is not particularly limited, but is, for example, egg of chicken, quail, duck, ostriche, and pigeon, preferably egg of chicken (hen's egg). In the present invention, the egg is unfertilized egg.

In the present invention, non-lethal animal-derived component derived from egg is not particularly limited, but is, for example, egg yolk, egg white, egg white albumin, egg yolk lecithin, and shell membrane.

In the present invention, a material of the edible base material is not particularly limited, but it is preferable not to use animal-derived material. In the present invention, the edible base material contains, for example, natural polymer polysaccharides such as alginic acid or alginate, glucomannan, cellulose derivative, amylose, pectin, glucomannan, agarose, carrageenan, and locust bean gum, microorganism producing polysaccharides such as bacterial cellulose, xanthan gum, gellan, pullulan, and hyaluronic acid, and microorganism producing polyamino acids such as polyglutamic acid and polylysine, preferably contains alginic acid or alginate, glucomannan, or cellulose derivative.

In the present invention, alginate is, for example, a salt of alginic acid and a divalent metal ion. For example, in alginate, at least one G-block contained in the alginic acid forms an ionic bond with a divalent metal ion. In other words, in the alginate contained in foam, the alginic acid forms a partial salt with the divalent metal ion. The alginate, for example, have a cross-linked structure mediated by divalent metal ion. The divalent metal ion includes calcium ion, barium ion, iron ion, zinc ion, copper ion, aluminum ion, etc. and calcium ion is preferred.

In the present invention, the edible base material may further contain other polysaccharide (P) other than alginic acid and alginate, and it is preferable to contain two or more other polysaccharides (P). In the foam, the two or more other polysaccharides (P) may associate with each other. The other polysaccharide (P), for example, acts as a foaming agent when the foam is made. The foam contains, as other polysaccharide (P), for example, at least one selected from the group consisting of glucomannan (konjak mannan) and cellulose derivative, preferably contains both glucomannan and cellulose derivative.

In the present invention, glucomannan is a polysaccharide contained in konjak root, etc. and has structural unit derived from glucose (glucose unit) and that derived from mannose (mannose unit). In glucomannan, each structural unit is linked mediated by 1,4-glycoside bond. In the glucomannan, the molar ratio of mannose units to glucose units is not particularly limited, but is, for example, 0.5 to 2 or may be 0.5 to 1.6.

In the present invention, cellulose derivative has a structure in which a substituent has been introduced into cellulose. The substituent preferably functions as a hydrophobic group in the cellulose derivative. The cellulose derivative includes, for example, cellulose ether. The cellulose ether includes, for example, alkyl cellulose such as methyl cellulose (MC): hydroxyalkyl cellulose such as hydroxypropyl cellulose (HPC) and hydroxyethyl cellulose (HEC): hydroxyalkyl alkyl cellulose such as hydroxypropyl methyl cellulose (HPMC): carboxyalkyl cellulose such as carboxymethyl cellulose (CMC). The cellulose derivative preferably contains hydroxypropyl methyl cellulose.

In one embodiment of the present invention, the edible base material is, for example, a porous material including multiple pores. The multiple pores, for example, are continuously formed in three dimensions, and, for example, has multiple continuous pores. However, the edible base material may further have independent pore in addition to the continuous pores.

In one embodiment of the present invention, the edible base material is not particularly limited, but is, for example, a non-woven fabric, perforated sheet, foam, etc., having multiple pores.

Average pore diameter of the pores included in the edible base material is not particularly limited, but is, for example, 50 μm to 2000 μm, preferably 50 μm to 1000 μm. For the average pore diameter of the edible base material, an area of a specific pore can be calculated by image processing in an electron microscope image of a cross section of the edible base material, and a diameter of a circle having the same area as the calculated area can be considered as the pore diameter (the diameter of the pore) of the specific pore.

Porosity of the edible base material is not particularly limited, but is, for example, 50% or more, preferably 70% or more, more preferably 80% or more. Upper limit for the porosity of the edible base material is not particularly limited, but is, for example, 99%. The porosity of the edible base material can be calculated, for example, by obtaining volume and weight of the edible base material to be evaluated and substituting the obtained volume and weight into the following formula (1). In the formula (1), V means volume (cm3), W means weight (g), and D means true density of the edible base material (g/cm3). The true density can be calculated, for example, from volume and weight of a solid obtained by performing steps (i) to (iv) without whisking (without foaming) solution (S) in the abovementioned production method. The true density can also be calculated based on gravity of each component contained in the edible base material.

Porosity (%)=100×[V−(W/D)]/V (1)

In the present invention, shape of the edible base material is not particularly limited, but can be appropriately adjusted according to shape of a cultured meat to be produced. As an example, the edible base material may be a sheet shaped or cube shaped with a thickness of 1 to 30 mm, or irregularly shaped.

Apparent density of the edible base material is not particularly limited, but is, for example, 0.8 g/cm³ or less, preferably 0.5 g/cm³ or less, more preferably 0.3 g/cm³ or less, and further more preferably 0.1 g/cm³ or less. Lower limit of the apparent density is not particularly limited, but is, for example, 0.01 g/cm³. The apparent density of the base material can be calculated from volume and weight of the base material to be evaluated.

In the present invention, “adhesion improver” refers to an agent to improve adhesion of cells and base material. In the present invention, the adhesion improver may, for example, be laminated onto the base material or may be coated on the base material. In addition, the adhesion improver may be mixed with the component comprising the base material during production of the base material to be integrated with the base material.

One aspect of the present invention relates to a cell culturing scaffold material containing the edible base material for cell culturing and the adhesion improver containing the edible and non-lethal animal-derived component of the present invention. In the present invention, the method of applying the adhesion improver in the cell culturing scaffold material is not particularly limited, but a layer containing the adhesion improver may be laminated on the surface of the edible base material, or the adhesion improver may be kneaded into the edible base material. In a embodiment of the present invention, the cell culturing scaffold material of the present invention has a layer that said adhesion improver is laminated on the surface of the edible base material.

In addition, if the layer containing adhesion improver is laminated, a strong film may be formed by gelling during drying, or a fragile film may be formed without gelling.

In the present invention, thickness of the layer containing said adhesion improver is not particularly limited, but is, for example, 1 nm or more. The thickness of the layer containing said adhesion improver is not particularly limited, but is, for example, 500 μm or less, 100 μm or less, 10 μm or less, 5 μm or less, 3 μm or less, 1 μm or less, 500 nm or less, or 300 nm or less. If the thickness of the layer containing said adhesion improver exceeds 500 μm, texture is affected.

Weight of the adhesion improver relative to that of the edible base material is not particularly limited, but is 0.01% to 50%, preferably 0.1% to 30%, more preferably 1% to 10%. In addition, the adhesion improver only needs to contribute to cell adhesion to the cell culturing base material, and the adhesion improver may remain on the edible base material while the cells are cultured on the cell culturing scaffold material, or part or all may outflow or elute into culture medium.

One aspect of the present invention relates to a tissue body in which cells are cultured on the cell culturing scaffold material of the present disclosure. In the present invention, cells to be cultured on the cell culturing scaffold material are not particularly limited, but are, for example, stem cells, progenitor cells, stromal cells, muscle progenitor cells, fibroblasts, pericytes, endothelial cells (such as aortic endothelial cells and skeletal microvascular endothelial cells), smooth muscle cells, fat cells, etc., preferably smooth muscle cells, fibroblasts, fat cells and stem cells, most preferably smooth muscle cells and fibroblasts.

In the present invention, the term “stem cells” as used herein includes mesenchymal stem cells (MSCs), embryonic stem cells (ESCs), adult stem cells, differentiated ESCs, differentiated adult stem cells, and induced pluripotent stem cells (iPSCs). The term “progenitor cells” as used herein refers to cells that can produce differentiated cells in multiple lineages, such as myoblasts, fibroblasts, fat cells, stromal cells, fibroblasts, and pericytes, smooth muscle cells, and endothelial cells. “Progenitor cells” typically differ from stem cells in that they do not have extensive self-renewal capabilities.

In one embodiment of the present invention, cells are derived from stem cells such as pluripotent embryonic stem cells. In one embodiment of the present invention, cells are mesenchymal stem cells (MSCs). As is known in the art, MSCs can be muscle cells, fat cells, osteocytes, and chondrocytes. In another embodiment of the present invention, cells are induced pluripotent stem cells (iPSCs). In yet another embodiment of the present invention, the cells are derived from totipotent embryonic stem cells such as cells from the blastocyst stage of animals, fertilized egg, placenta, or umbilical cord of animals.

In one embodiment of the present invention, the cells are progenitor cells. In one embodiment of the present invention, the cells are progenitor cells of myoblasts or those of fat cells.

In one embodiment of the present invention, progenitor cells are cultured in monoculture. In one embodiment of the present invention, progenitor cells are differentiated in monoculture. In one embodiment of the present invention, progenitor cells are differentiated in monoculture and then seeded on a cell culturing scaffold material that is incubated with multiple cells according to the method of the present invention. In one embodiment of the present invention, mesenchymal stem cells are cultured and differentiated to myoblasts, and then the differentiated myoblasts are seeded onto the cell culturing scaffold material and subsequently incubated. Methods for culturing progenitor cells and inducing differentiation into mature cells are known in the art.

In one embodiment of the present invention, cells are obtained from living animals and cultured as primary cell lines. In other embodiment of the present invention, cells may be obtained by biopsy or may be cultured ex vivo. In yet another embodiment of the present invention, cells are obtained from commercial sources.

In one embodiment of the present invention, the cell culturing scaffold material of the present invention is a scaffold for culturing muscle progenitor cells in vitro or ex vivo, and for differentiating muscle progenitor cells into specific type of muscle cells such as skeletal muscle cells or smooth muscle cells. In addition, the cell culturing scaffold material of the present invention may be used to culture fibroblasts, which can secrete extracellular molecules that form an extracellular matrix (ECM) that provides additional structural and mechanical support to the cells.

In one embodiment of the present invention, the cell culturing scaffold material of the present invention may be used for culturing fat cells, which can provide a specific flavor and texture.

In one embodiment of the present invention, the cell culturing scaffold material may be used to culture endothelial cells such as aortic endothelial cells and skeletal microvascular endothelial cells, or capillary endothelial formed by the endothelial cells.

In the present invention, the cell culturing scaffold material may be sterilized prior to seeding or incubating cells. In the present invention, the sterilization is not particularly limited, but is performed, for example, by ethanol or by gamma irradiation.

In the present invention, cell seeding and/or culturing is performed in the presence of cell culture medium, which may contain growth factor, cytokine, bioactive agent, nutrient, amino acid, antibiotic compound, anti-inflammatory compound, or any combination thereof.

In one embodiment of the present invention, the cell culturing scaffold material of the present invention may contain a composition that is suitable for cell growth. The composition that is suitable for cell growth is not particularly limited, but include, for example, growth factor, cytokine, bioactive agent, nutrient, amino acid, antibiotic compound, anti-inflammatory compound, natural dye, perfume, etc.

The growth factor that can be used in the present invention is not particularly limited, but include, for example, platelet-derived growth factor (PDGF), insulin-like growth factor (IGF-1), etc. The growth factor is not particularly limited, but includes, PDGF (e.g., PDGF AA, PDGF BB): IGF (e.g., IGF-I, IGF-II): fibroblast growth factor (FGF) (e.g., acidic FGF, basic FGF, beta-endothelial cell growth factor, FGF 4, FGF 5, FGF 6, FGF 7, FGF 8, and FGF 9): transforming growth factor (TGF) (e.g., TGF-P1, TGFβ1.2, TGF-β2, TGF-β3, TGF-B5): bone morphogenetic protein (BMP) (e.g., BMP 1, BMP 2, BMP 3, BMP 4): vascular endothelial growth factor (VEGF) (e.g., VEGF, placental growth factor): epidermal growth factor (EGF) (e.g., EGF, amphiregulin, betacellulin, heparin-binding EGF): interleukin (e.g., IL-1, IL-2, IL-3, IL-4, IL-5, IL-6, IL-7, IL-8, IL-9, IL-10, IL-11, IL-12, IL-13, IL-14): colony stimulating factor (CSF) (e.g., CSF-G, CSF-GM, CSF-M): nerve growth factor (NGF): stem cell factor: hepatocyte growth factor, and ciliary neurotrophic factor, etc.

In the present invention, the cells cultured on the cell culturing scaffold material are not particularly limited, but are cells derived from mammalian animals, avian animals, fish animals, reptilian animals, amphibian animals, and invertebrate animals (such as crustaceans). In the present invention, cells of mammalian animals include cells of cattle, pigs, sheep, horses, bears, goats, rabbits, antelopes, bison, boars, whales, dolphins, beavers, sea otters, dugongs, manatees, seals, sea lions, walruses, weasels, camels, reindeer, deer, elephants, elks, foxes, giraffes, ibex, kangaroos, lions, llamas, moose, peccaries, squirrels, tigers, yaks, zebras, etc.

In the present invention, cells of avian animal include cells of chickens, ducks, turkeys, emus, geese, grouse, ostriches, pheasants, pigeons, quail, etc.

In the present invention, cells of fish animal include cells of tunas, sharks, rays, anglerfishes, sunfishes, marlins, mackerels, horse mackerels, bonitos, sea basses, mackerels, red sea breams, flatfishes, flounders, eels, herrings, salmons, killer whales, sardines, red snappers, blow fishes, basses, catfishes, carps, cods, groupers, haddocks, halibuts, herrings, mahi, swordfishes, orange roughies, perches, pikes, Alaska pollocks, sardines, snappers, swordfishes, tilapias, trouts, walleyes, etc.

In the present invention, cells of reptilian animal include cells of snakes, alligators, turtles, etc. In the present invention, cells of amphibian animal include cells of frogs, etc.

In the present invention, cells of crustacean animal include cells of shrimps, crabs, krills, hermit crabs, crayfishes, lobsters, etc

In the present invention, cells of other invertebrate animal include cells of clams, abalones, seahares, oysters, turban shells, freshwater clams, hard clams, scallops, soletellina diphos, mussels, sea urchins, ascidians, etc.

In the present invention, cells are seeded at a cell density that is favorable for their growth. In one embodiment of the present invention, cells are seeded simultaneously or consecutively. In one embodiment of the present invention, cells are seeded at a cell density of 10³ to 10⁷ cells/cm². In one embodiment of the present invention, a cell density when seeded and the one when incubated are approximately the same.

In one embodiment of the present invention, coverage of cells on the surface of the cell culturing scaffold is at least 5%, at least 20%, at least 35%, at least 50%, at least 70%, at least 85%, at least 90%, at least 95%, or at least 99%.

In one embodiment of the present invention, cells are seeded so that the coverage of cells on the surface of the cell culturing scaffold to be 1 to 10%, 5 to 20%, 15 to 35%, 30 to 50%, 40 to 65%, 60 to 85%, 80 to 90%, 90 to 100%, or any range between thereof.

In one embodiment of the present invention, the tissue body is for food. In one embodiment of the present invention, the tissue body of the present invention is intended for consumption by human, non-human animal, or both of them, preferably by human. In other embodiment of the present invention, the tissue body of the present invention is intended for consumption by non-human animal, and is used as, for example, animal feed such as livestock feed, aquaculture feed, or household pet feed.

In the following, the present invention will be described in more detail with examples, but the present invention is not limited thereby.

### EXAMPLES

### 1. Preparation of Base Material

Needling was performed on alginate wound dressing (Sorbsan Flat No. 1, 50 mm×50 mm (made by ALCARE)) using a felt puncher (made by Crobar or Fujikyu) set with two needles, to enhance fiber entwinement. The needling was performed on a 28.5 mm square alginate wound dressing by inserting and removing two needles several times per site, at a pitch of 1.5 mm, for a total of 400 sites. The alginate wound dressing on which needling was performed was immersed in a sodium alginate solution adjusted to 0.1%. The alginate wound dressing was then removed and thoroughly wiped dry. It was then immersed in a 100 mM calcium chloride solution for 5 minutes to gelate alginate. The alginate wound dressing was then removed, thoroughly wiped dry, and dried at 90° C. for 1 hour. This was used as the base material for tests.

### 2. Preparation and Coating of an Adhesion Improver

Aqueous solutions or aqueous dispersions containing various edible and non-lethal animal-derived materials at the concentrations listed in Table 1 were prepared. The base material prepared in example 1 was immersed in the prepared aqueous solution or aqueous dispersion for 5 minutes. The base material was then removed, thoroughly wiped dry, and dried at 90° C. for 1 hour.

TABLE 1

| | Coating | | |
|---|---|---|---|
| | | | Content |
| | Material | Product name/Manufacturer | (wt %) |
| Example 1 | Whey protein A | Genesis A/DAIICHI-KASEI | 0.10% |
| Example 2 | Whey protein A | Genesis A/DAIICHI-KASEI | 1.00% |
| Example 3 | Whey protein A | Genesis A/DAIICHI-KASEI | 0.01% |
| Example 4 | Whey protein B | Daiichirakuto EM-90/ | 0.10% |
| | | DAIICHI-KASEI | |
| Example 5 | Casein sodium A | Caserone SD/DAIICHI- | 0.10% |
| | | KASEI | |
| Example 6 | Casein sodium B | Caserone L/DAIICHI-KASEI | 0.10% |
| Example 7 | Casein sodium C | Sunlact S-3/Taiyo Kagaku | 0.10% |
| Example 8 | Egg white | | 1.00% |
| Example 9 | Egg yolk | | 1.00% |
| Example 10 | Egg white peptide | Peptide EP-1/Kewpie | 0.10% |
| Example 11 | Egg yolk lecithin | PL-30S/Kewpie | 0.10% |

Whey proteins A and B used in the experiment are further processed isolated whey protein in which milk sugar and mineral have been removed from whey. In the experiment, Genesis A (DAIICHI-KASEI) was used as whey protein A, which contains 91% of protein, 0% of fat, 7% of carbohydrate, and 3% of ash and mineral. In the experiment, Daiichirakuto EM-90 (DAIICHI-KASEI) was used as whey protein B, which contains 87.6% of protein, 3.1% of fat, 0% of carbohydrate, and 5% of ash and mineral.

Egg white and egg yolk were respectively used by separating commercially available hen's egg into egg white and egg yolk.

In the experiment, Casein SD (DAIICHI-KASEI), which is powdered casein sodium with spray-dry method, was used as casein sodium A, Casein L (DAIICHI-KASEI), which is powdered casein sodium with extruder method, was used as casein sodium B, and Sunlact S-3 (Taiyo Kagaku) was used as casein sodium C.

In the experiment, Peptide EP-1 (Kewpie) was used as egg white peptide, which contains 85.9% of protein, 0.1% of fat, 4.3% of carbohydrate, and 12% of ash and mineral. In the experiment, PL-30S1 (Kewpie) was used as egg yolk lecithin, which contains 0% of protein, 97% of fat, 0% of carbohydrate, and 1% of ash and mineral.

Base material coated with various edible and non-lethal animal-derived material was stamped out to make test specimen of 6 mm diameter disk.

### 3. Culture Test

Specimens were sterilized by immersing in a 70 w/w % ethanol solution and allowing them to stand for 30 minutes. Ethanol was then removed by washing them three times with ultrapure water. The specimens were placed in a 96-well plate (Nunc (trademark) MicroWell (trademark) 96-Well #167008) and each 100 μL of culture medium (DMEM (High glucose) D6546 (Sigma)) in which final concentration of 4 mM L-Glu (L-Glu 25030081 (Thermo)), 10% FBS (FBS 10270 (Thermo)), and 100 units/mL penicillin streptomycin (#168-23191 (Wako)) was respectively supplemented were added, then the plate was allowed to stand for 15 minutes. Culture medium that was not soaked were then removed. Then, a cell solution containing NIH3T3 cells, mouse fetus skin cells, at a density of 1×10⁵ to 1×10⁷ cells/cm² was slowly dropped and allowed to stand for 30 minutes. Then, sufficient amount of culture medium was supplemented to soak the base material, and culture was started under the condition of 5% CO² and 37° ° C. One day later, the base materials were moved to a new plate, and the culture medium were replaced every 3 days for a total of 7 days of continued culture at 5% CO² and 37° ° C.

Also, a culture test on the egg white itself was performed. Commercially available hen's egg was separated into egg white and egg yolk, 1% w/w of sugar was added to the egg white, which was whisked with an electric mixer for 5 minutes. The whipped egg white was poured into a mold with about 6 cm diameter, processed at 100° C. for 1 hour to dry substantially, and culture base material was produced by punching out the dried egg white with a 6 mm diameter biopsy punch and cultured in the same manner (Example 12).

Further, similar culture tests were performed for collagen sponge (honeycomb type CSH-96 (KOKEN)) (referred to Reference Example 1), the base material prepared in 1. (referred to Comparative Example 1), and casein-containing fiber (referred to Comparative Examples 2), each of which are without coating of edible and non-lethal animal-derived component.

Collagen sponge is processed and lyophilized atelocollagen, for which collagen sponge (honeycomb CSH-96 (KOKEN)) was used.

Casein-containing fiber was cut out from fiber of Day Towel Natural 25×90 cm Bless. Milk BL-402 (FUJIEI), and stuffed into wells without needling.

### 4. Cell Number Evaluation by CTG Assay

After three days of culture, 100 μL of cell number evaluation reagent (CellTiter-Glo (registered trademark) 2.0 Cell Viability Assay G9243 (Promega)) was added to each well and gently stirred for about 2 minutes. 200 μL of supernatant from each well was moved to a 96-well plate for luminescence measurement (Perkin Elmer (trademark) OptiPlate-96 #6005299), luminescence was measured according to standard protocol for luminescence measurement of plate reader (Perkin Elmer EnSight), and cell number was calculated using previously prepared calibration curve. Cell number was measured with automated cell counter Thermo Countess (registered trademark) II FL. Calculated cell numbers were shown as percentage to the cell number calculated in the collagen sponge (honeycomb type CSH-96 (KOKEN)) (Reference Example 1).

The results are shown in Tables 2.

TABLE 2

| | | | | | Cell |
|---|---|---|---|---|---|
| | Substrate | Coating | | | number |
| | material | | Product name/ | Content | (%) (To |
| | Material | Material | Manufacturer | (wt %) | collagen) |
| Example 1 | Alginic non- | Whey | Genesis A/ | 0.10% | 18% |
| | woven fabric | proteins A | DAIICHI-KASEI | | |
| Example 2 | Alginic non- | Whey | Genesis A/ | 1.00% | 22% |
| | woven fabric | proteins A | DAIICHI-KASEI | | |
| Example 3 | Alginic non- | Whey | Genesis A | 0.01% | 39% |
| | woven fabric | proteins A | DAIICHI-KASEI | | |
| Example 4 | Alginic non- | Whey | Daiichirakuto EM- | 0.10% | 20% |
| | woven fabric | proteins B | 90/DAIICHI- | | |
| | | | KASEI | | |
| Example 5 | Alginic non- | Casein | Caserone SD/ | 0.10% | 36% |
| | woven fabric | sodium A | DAIICHI-KASEI | | |
| Example 6 | Alginic non- | Casein | Caserone L/ | 0.10% | 34% |
| | woven fabric | sodium B | DAIICHI-KASEI | | |
| Example 7 | Alginic non- | Casein | Sunlact S-3/Taiyo | 0.10% | 41% |
| | woven fabric | sodium C | Kagaku | | |
| Example 8 | Alginic non- | Egg white | | 1.00% | 23% |
| | woven fabric | | | | |
| Example 9 | Alginic non- | Egg yolk | | 1.00% | 27% |
| | woven fabric | | | | |
| Example 10 | Alginic non- | Egg white | Peptide EP-1/ | 0.10% | 8% |
| | woven fabric | peptide | Kewpie | | |
| Example 11 | Alginic non- | Egg yolk | PL-30S (Kewpie) | 0.10% | 7% |
| | woven fabric | lecithin | | | |
| Example 12 | Egg white | No coating | | | 12% |
| Comparative | Alginic non- | No coating | | | 6% |
| Example 1 | woven fabric | | | | |
| Comparative | Casein- | No coating | | | 0% |
| Example 2 | containing | | | | |
| | fiber | | | | |

A significant increase in the number of cells cultured was observed when cells were cultured on the scaffold coated with edible and non-lethal animal-derived component on the base material (Examples 1 to 11) and when cells were cultured on egg white itself as scaffold (Example 12), compared to when cells were cultured on the base material without coating edible and non-lethal animal-derived component (Comparative Example 1) and when cells were cultured on casein-containing fiber as the scaffold (Comparative Examples 2).

5. Morphological Observation with Confocal Microscope

After 7 days of culture, cells were stained with live cell staining reagent (Calcein AM 1 mg/mL in DMSO #349-07201 (Wako)) and nuclear staining reagent (Hoechst 33342 10 mg/mL in H2O #H3570 (Thermo)) and morphology was observed with confocal microscope (Nikon A1 plus). In two-dimensional image, transmitted light image, Calcein AM stained image (green), and Hoechst stained image (blue) were superimposed, and in three-dimensional image, autofluorescence image (red) was superimposed instead of the transmitted light image.

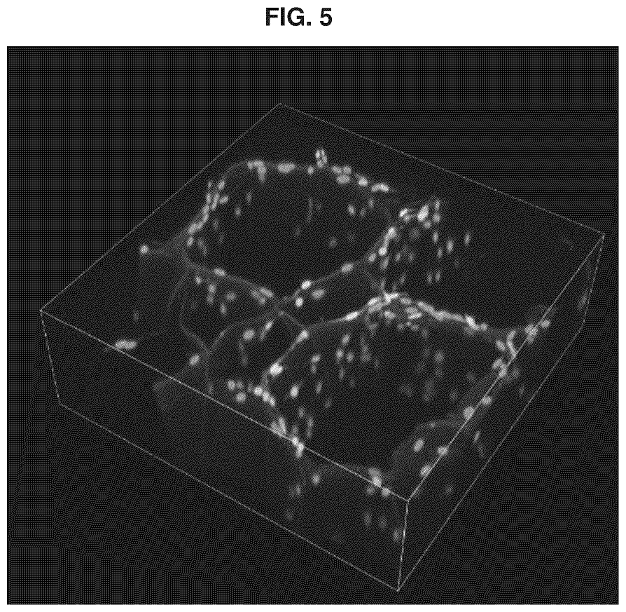

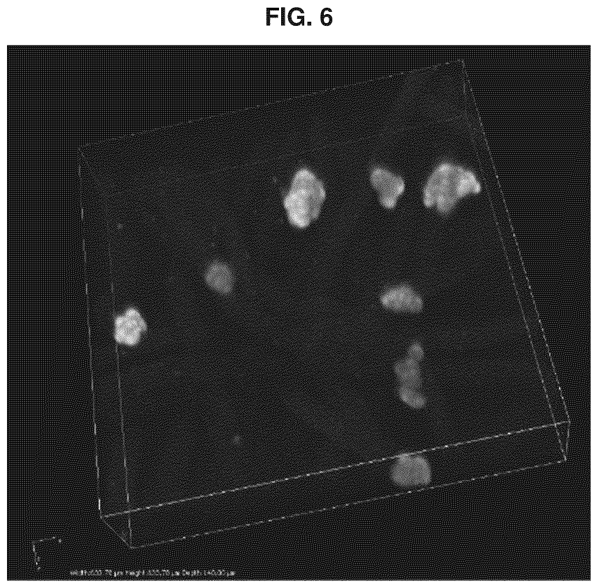

Two-dimensional images and three-dimensional images of Examples 1 to 4, and three-dimensional images of Reference Example 1 and Comparative Example 1 are shown in FIGS. 5 and 6, respectively.

In Comparative Examples 1, the cells did not adhere to the base material, and the cells gathered together to form cell aggregation. Cells cannot grow under this situation.

In Examples 1 to 2, cells adhered to the base material and cytoplasm extended well, confirming cell growth. In Examples 3 to 4, cells adhered to the base material, but extension of cytoplasm was a little less.

### 6. Coating Thickness Measurement Test

Coating thickness of coating layer of the specimens in Comparative Example 1, Example 7 and Example 8 were measured by TOF-SIMS, and coating thickness of coating layer of the specimens in Comparative Example 1, Examples 7, Example 8, Example 13 and Example 14 were measured by SEM.

TRIFT-V (ULVAC-PHI) was used for TOF-SIMS. The specimens were cut into 1 cm square, fixed onto a sample table, irradiated with Ar gas cluster ions (Arₙ⁺) as etching ion (etching ion acceleration voltage: 20 kV), irradiated with Bi₃²⁺ as primary ion (primary ion acceleration voltage: 30 kV), and the coating thickness was measured over a measurement area of 500 μm square. A neutralizing gun for charge correction was used for the measurement.

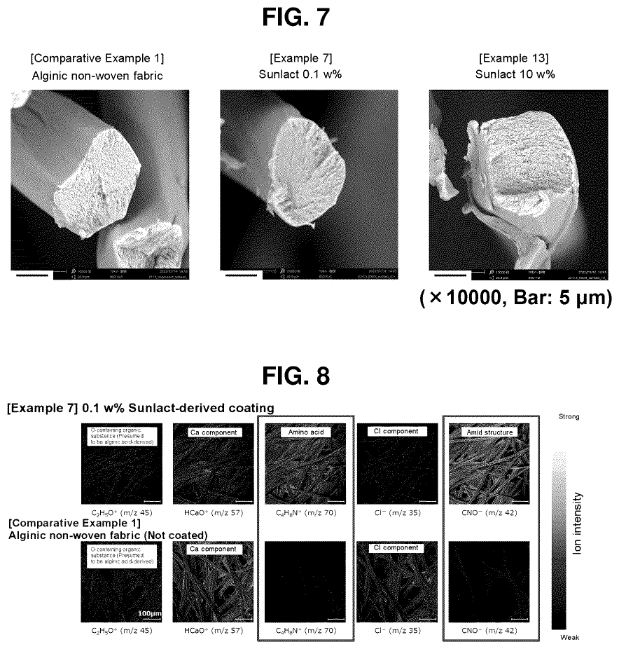

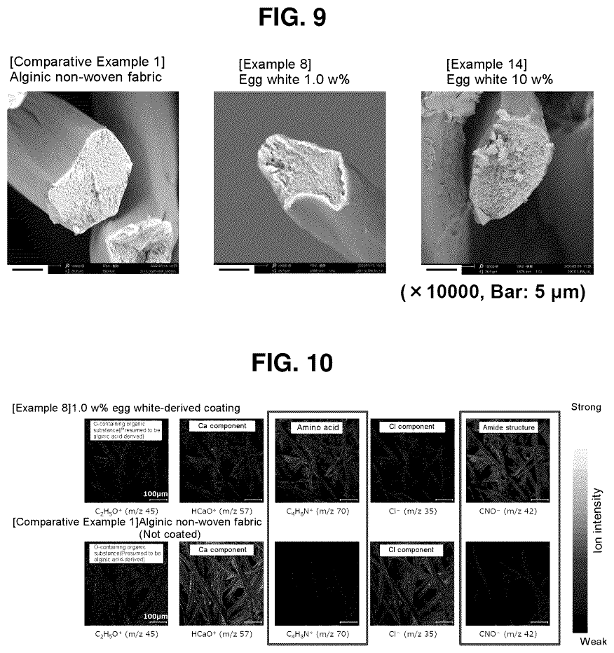

The results are shown in Tables 3. Also, SEM images of specimens in Comparative Example 1, Example 7, and Example 13 are shown in FIG. 7. TOF-SIMS images of the specimens in Comparative Example 1 and Example 7 are shown in FIG. 8. SEM images of specimens in Comparative Example 1, Example 8, and Example 14 are shown in FIG. 9. TOF-SIMS images of the specimens in Comparative Example 1 and Example 8 are shown in FIG. 10.

Since no amino acid was detected by TOF-SIMS in Comparative Example 1, on the other hand, amino acid was detected by TOF-SIMS in Examples 7 and 8, the coating thickness is considered to be 1 nm or more. However, in Examples 7 and 8, the layer thickness is considered to be below the SEM resolution of 34 nm, because definite layer could not be identified by SEM.

TABLE 3

| | | | | | Coating thickness (nm) | | |
|---|---|---|---|---|---|---|---|
| | Substrate | Coating | | | | SEM (Average | SEM (Max. |
| | material | | Product name/ | | TOF- | coating | coating |
| | Material | Material | Manufacturer | Content | SIMS | thickness) | thickness) |
| Example 7 | Alginic | casein | Sunlact S-3/ | 0.10% | 1 or | — | 34 or |
| | non-woven | sodium C | Taiyo Kagaku | | more | | less |
| | fabric | | | | | | |
| Example 13 | Alginic | casein | Sunlact S-3/ | 10.00% | — | 1330 | 3065 |
| | non-woven | sodium C | Taiyo Kagaku | | | | |
| | fabric | | | | | | |
| Example 8 | Alginic | Egg white | | 1.00% | 1 or | — | 34 or |
| | non-woven | | | | more | | less |
| | fabric | | | | | | |
| Example 14 | Alginic | Egg white | | 10.00% | — | 834 | 1370 |
| | non-woven | | | | | | |
| | fabric | | | | | | |

1. An adhesion improver containing edible and non-lethal animal-derived component, that is used in an edible base material for cell culturing.

2. The adhesion improver according to claim 1, wherein the edible and non-lethal animal-derived component is derived from milk or egg.

3. The adhesion improver according to claim 2, wherein the milk is cow's milk.

4. The adhesion improver according to claim 1, wherein the edible and non-lethal animal-derived component is casein or whey.

5. The adhesion improver according to claim 2, wherein the egg is a hen's egg.

6. The adhesion improver according to claim 1, wherein the edible and non-lethal animal-derived component is egg yolk or egg white.

7. The adhesion improver according to claim 1, wherein the edible base material contains alginic acid or alginate, glucomannan, or cellulose derivative.

8. The adhesion improver according to claim 1, wherein the edible base material is a porous material.

9. A cell culturing scaffold material containing the edible base material for cell culturing and the adhesion improver according to claim 1.

10. The cell culturing scaffold material according to claim 9, wherein a layer containing said adhesion improver is laminated on a surface of the edible base material.

11. The cell culturing scaffold material according to claim 10, wherein thickness of the layer containing said adhesion improver is 1 nm or more.

12. A tissue body in which cells are cultured on the cell culturing scaffold material according to claim 9.

13. The tissue body according to claim 12, wherein the cells are derived from mammal, fish, or crustacean.

14. The tissue body according to claim 12, wherein the tissue body is for food.
